(19) United States
(12) Patent Application Publication
(10) Pub. No.: US 2017/0273554 A1
(43) Pub. Date: Sep. 28, 2017

(54) AN OPTICAL COHERENCE TOMOGRAPHY SYSTEM AND METHOD

(71) Applicant: COSTRUZIONI STRUMENTI OFTALMICI C.S.O. S.R.L., Scandicci (Firenze) (IT)

(72) Inventors: Silvano PIERI, Sesto Fiorentino (Firenze) (IT); Gabriele VESTRI, Firenze (IT); Matteo MARCACCI, Capraia e Limite (Firenze) (IT); Claudio DE MAURO, Siena (IT); Domenico ALFIERI, Lastra a Signa (Firenze) (IT); Francesco VERSACI, Prato (IT); Simone SPADINI, Firenze (IT)

(21) Appl. No.: 15/506,798

(22) PCT Filed: Sep. 2, 2014
(86) PCT No.: PCT/IB2014/064201

§ 371 Date: Feb. 27, 2017

(51) Int. Cl. A61B 3/10; G01B 9/02

(52) U.S. Cl. CPC A61B 3/102; G01B 9/02028; G01B 9/02044; G01B 9/02058; G01B 9/02063; G01B 9/02064; G01B 9/02091

(57) ABSTRACT

The present invention relates to the field of instruments for imaging internal structures of the human body, and in particular of the eye. More specifically it relates to an optimized process and an optical coherence tomography system thereof to measure the distances between the eye interfaces that is, the corneal surfaces, the surfaces of the crystalline lens, the retina and so on. A tiltable selection means, e.g. a titable mirror, is used to switch between different optical sample paths having different lengths, such that information relative to portions of the sample at different depths can be collected.

### FIELD OF THE INVENTION

The present invention relates to the field of instruments for imaging internal structures of the human body, and in particular of the eye. More specifically it relates to an optimized process and an optical coherence tomography system thereof to measure the distances between the eye interfaces (that is, the corneal surfaces, the surfaces of the crystalline lens, the retina and so on).

### BACKGROUND OF THE INVENTION

Optical coherence tomography (OCT), also referred to as phase-variance optical coherence tomography, is one of the most powerful and most widespread biomedical imaging techniques. It has applications in several fields of medicine. The ophthalmologic field has greatly contributed to its development and optimization.

In this technique any information relating to the structure of the sample/organ being observed is derived from the radiation reflected back and/or backscattered from regions showing different optical properties within the sample/organ itself.

The OCT technique allows to create two-dimensional or three-dimensional models having a resolution of one to few pm. Besides allowing a morphological study, OCT may reveal other biological properties of the sample being analysed, such as for example flow rate (by means of the Doppler effect) and birefringence (by means of polarisation changes).

OCT has its foundations in low-coherence interferometry. The optical set up of the OCT system is based on a Michelson interferometer and the OCT system operating mode is determined depending on the type of radiation source and detection technique used. Currently, there are two main schemes used in OCT instruments.

In the so-called Time-Domain OCT (TD-OCT) the reflectivity profile of the sample is obtained by having the radiation coming from the sample optical arm interfere with that coming from the reference optical arm, whose path is modified within a certain time interval. The displacement of the reference arm is the measurement of the distance of the sample member that has caused the reflection.

The Fourier Domain OCT (FD-OCT), on the contrary, records in one step, without the need of a mechanical translation of the members in the reference arm, the spectrum fringes caused by the interference of the radiation coming from the sample arm with that coming from the reference arm, in a broad spectral band. The measurement of the distances of the various sample members is obtained by processing the interferogram signal.

The second technique is much faster than the first one in that it reduces the presence of moving parts and also has benefits in terms of signal-to-noise ratio which result in higher image quality.

In turn, the second FD-OCT technique may be applied according to two main embodiments:
Spectral Domain OCT (SD-OCT), wherein the spectrum is obtained by using a broadband radiation source and a spectrometer which measures its intensity with a linear sensor (line-scan camera);
Swept Source OCT (SS-OCT), wherein the spectrum is obtained by an individual radiation detector by making the wavelength emitted by the source vary at very high speeds.

In order to clarify the concepts, hereinafter reference will be made to a configuration of the SD-OCT type, but with obvious adjustments the man skilled in the art may readily extend the technique that will be illustrated to the other configurations referred to hereinabove and to known variations thereof.

With specific reference now to FIG. 1, which relates to a conventional SD-OCT configuration, the system provides:
a broadband radiation source LBS;
a reference optical arm RA which contains a lens system L2 and a mirror Mref;
a sample arm SA which contains a scanning system, consisting of a lens system L1 and a mirror and actuator system M, which allows to illuminate a strip (in the axial direction) of the sample of which an image is to be generated and the backscattered radiation is to be collected;
a signal detection arm MA with a spectrometer Spec which allows to analyse the spectrum of the signal resulting from the interference of the radiation coming from the reference arm RA and from the sample arm SA, comprising a linear sensor detecting the spectrum of the interference signal corresponding to the illuminated strip of the sample;
a beam-splitter BS configured so that it allows the passage of the radiation from the source LBS to the sample arm SA and to the reference arm RA, and from these to the detection arm MA; and
a control and processing unit CUP which suitably controls the mechanical and electronic components, and derives from the spectrum, by means of one of the many algorithms known in the literature, a reflectivity profile of the sample strip an image of which is to be generated.

The broadband light radiation source LBS is transmitted to the reference arm RA and to the sample arm SA opposite to which the sample to be imaged is placed. The radiation in the reference arm RA is reflected by the mirror MRef and is sent through the beam-splitter BS to the detection arm MA. Similarly, the radiation in the sample arm SA is backscattered from the illuminated sample portion and arrives through the beam-splitter BS to the detection arm MA. Therefore, the two light waves, coming from the reference arm RA and the sample arm SA, interfere with the detection arm MA where the spectrometer Spec reconstructs on a linear sensor the spectrum of the interference signal (interferogram).

The above-mentioned spectrum is transformed by means of one of the algorithms known in the literature in the reflectivity profile of the illuminated sample portion. If, for multiple strips (A-scans), it is possible to measure the reflectivity profile, a cutaway image (B-scan) of the sample may be obtained. From such a cutaway image measurements relating to the shape of the sample may be obtained. In the case of an eye, for example (see the illustration of FIG. 2), if the anterior eye segment is observed, the altimetrical profile and the curvature of the surfaces of the cornea, the crystalline lens and the iris may be obtained. If many images relating to different sample sections are captured, it may even be possible to generate a three-dimensional model of the sample.

If one decides to use a configuration according to the SS-OCT technique, the man skilled in the art may replace the broadband source with a source having an emitted wavelength that can be varied very quickly over time, and the spectrometer of the detection branch with a single detection channel radiation detector. In this case, the output signal spectrum is built by varying the wavelength emitted by the source and by sequentially storing the intensities measured by the detector for each wavelength.

In order to obtain an image of a section of the anterior eye segment, therefore a linear scan is generally performed and at the end the information obtained is processed into one single image. Then with reference to FIG. 3, if one assumes the use of just one mirror M for a two-dimensional scan, the scan is obtained by changing the inclination of the mirror in the sample arm and consequently the side position of the lighting beam coming from lens O. When the mirror is in position M′, the lighting beam R′ illuminates the central part of the scanning space and allows the detection of structures in that portion of the sample. When the mirror is in position M″, the lighting beam R″ illuminates the bottom part of the scanning space. When the mirror is in position M′″, the lighting beam R′″ illuminates the top part of the scanning space.

The illuminated tissue portion backscatters part of the radiation, with an angular scattering of the intensity that depends on its microstructure and the orientation of its discontinuity surfaces. In general such scattering, also referred to as lobe, will be uneven, with an intensity peak in the reflection direction, symmetrical to that of lighting as compared to the normal to said surfaces, and with decreasing intensity in the peripheral directions. The radiation that is actually collected for measurement is that which is backscattered exactly in the opposite direction to that of lighting. Such radiation, which returns to the instrument, will pass through the sample arm of the interferometer and will interfere in the detection arm with the radiation coming from the reference arm on the spectrometer branch.

A problem that may be found with the FD-OCT technology in its known variations is connected to the difficulty of capturing an image relating to a field of view deeper than about ten mm in air. Considering that the eye axial length in humans ranges approximately from 14 mm to 36 mm, from such difficulty there results the impossibility of generating a unique image containing a complete section of the eye from the cornea to the retina, unless one wants to use components significantly complicating the basic architecture of the system, which components moreover are still undergoing optimisation, whose effectiveness and reliability are still to be verified and whose costs are not commercially acceptable.

Among the examples of known solutions, those shown in the following patent documents may be reported.

U.S. Pat. No. 6,922,250 proposes a system for obtaining tomograms of the eye structure by means of a scan multiplex, based on low coherence interferometry, recorded simultaneously across points transversally adjacent in the pupil. Another task is to obtain a dynamic focusing so that the image captured scans the depth of the object in synchronism with the coherence window. Such results are achieved with a single path sample arm on which there is a moving mirror which, by moving longitudinally on an axis, varies the length of the arm in a continuous manner and shifts the focus of the scan at the desired capturing depth. This solution is not very robust against movements of the eye, if measurements of distances between eye structures present on the images captured at different depths are to be obtained.

EP1959816 describes a system with two reference arms, of which at least one is variable in length, and two beams coming from the sample, which are used according to a strategy based on which one of the beams simultaneously coming from the sample is used as the reference beam. The two beams coming from the sample are obtained by dichroic separation. A solution with a single reference arm with two mirrors, of which one is semi-transparent and the other is translatable, is also proposed. A sensor having a high number of photosensitive cells or pixels (costly and bulky) is then used by means of which the signal relating to an anterior eye structure and a posterior eye structure are captured in a single measurement. In any case, there is disclosed a complex structure from both the structural and operational standpoint. In particular, the continuous longitudinal movement of the end mirror of the reference arm used to shift the field of view in depth requires very high precision, without which the measurement accuracy may be jeopardised, but which may hardly be ensured due to vibrations, thermal expansions, frictions variable with wear.

Other documents wherein general reference is made to OCT systems suitable for capturing measurements deep in the eye structure, or at least for changing the focus along the axial direction of the above-mentioned structure, by adopting solutions associated entirely or in part to the preamble of the appended claim 1, are EP1713378, EP1781161, EP2346386 and U.S. Pat. No. 6,057,920.

### SUMMARY OF THE INVENTION

The present invention, on the other hand, proposes an efficient solution to the problem of obtaining acquisitions and measurements on a broad axial extension of a sample/organ such as an eye structure, employing an architecture configuration which is simple and as such may be carried out with relatively low costs and is very reliable from the operational point of view.

According to the invention, an optical coherence tomography system and method has the essential features referred to in the appended claims one and ten.

The basic idea of the invention is that of arranging on the sample arm a set of paths having different length selectable depending on the depth at which a section of the same sample is to be captured. Based on the images relating to different depths of the sample captured, on the recognition of the differences in length between the paths of the sample and reference arms, the distances between the surfaces of interest of the sample may be obtained. If the sample is in fact an eye, it is for example possible to identify the thickness of the cornea, the depth of the anterior chamber, the thickness of the crystalline lens and the distance of the cornea from the retina (axial eye length).

### BRIEF DESCRIPTION OF THE DRAWINGS

The features and the advantages of the optical coherence tomography process and system according to the present invention will appear more clearly from the following description of embodiments thereof, reported by way of a non-limiting example, with reference to the annexed drawings, wherein:

FIG. 1 is a representative scheme of an SD-OCT configuration;

FIG. 2 shows a complete cutaway image of the anterior segment of an eye reconstructed by matching individual scan strips with an OCT system;

FIG. 3 is a schematic representation of the scan operation on the sample arm of an OCT system;

FIG. 4 schematically shows a sample arm of an FD-OCT instrument according to the invention;

FIG. 5 is a further illustration of the mirror of the sample of FIG. 4 with operating selection of one of the mirrors provided therein;

FIG. 6 is yet a further illustration of the mirror of the sample of FIG. 4 with operating selection of another one of the mirrors provided therein;

FIG. 7 and FIG. 8 respectively show an anterior segment and a retina of an eye obtained according to the invention, respectively with the distance of the anterior corneal surface from the upper edge of the image and the distance of the retinal surface from the upper edge of the image schematised;

FIG. 9 is a representation analogous to those of FIG. 4 and FIG. 5 of a sample arm with curved mirrors to focus the scanning beam at the depths in accordance with the length of the various paths according to a different embodiment of the invention;

FIG. 10 is a representation analogous to those of FIG. 4 and FIG. 5 of a sample arm with dispersion compensator devices according to yet a different embodiment of the invention; and

FIG. 11 shows as in the preceding FIGS. 9 and 10 yet a further embodiment combining those of the above-mentioned FIGS. 9 and 10, that is, by adopting a sample arm with dispersion compensator devices and curved mirrors which focus the scanning beam in accordance with the operating depth of the various paths.

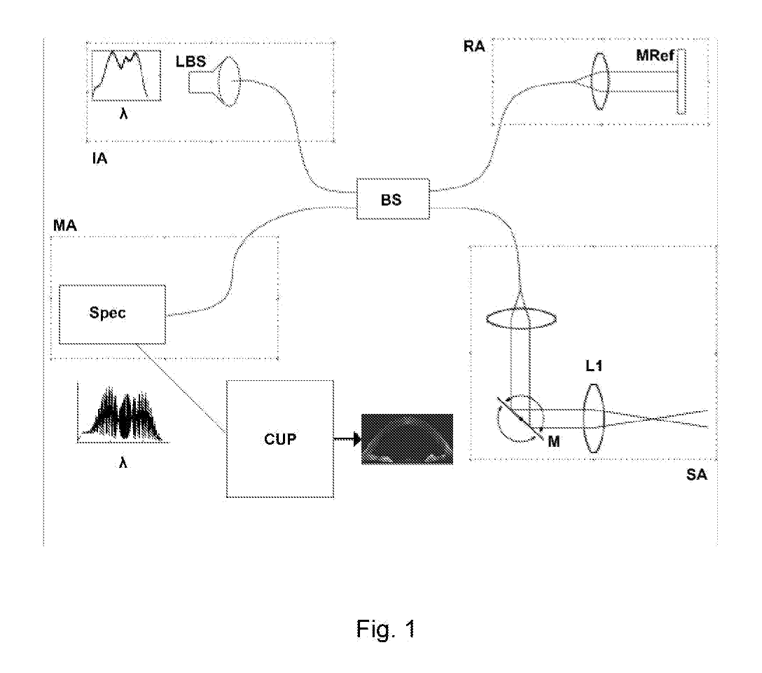

### DETAILED DESCRIPTION OF THE INVENTION

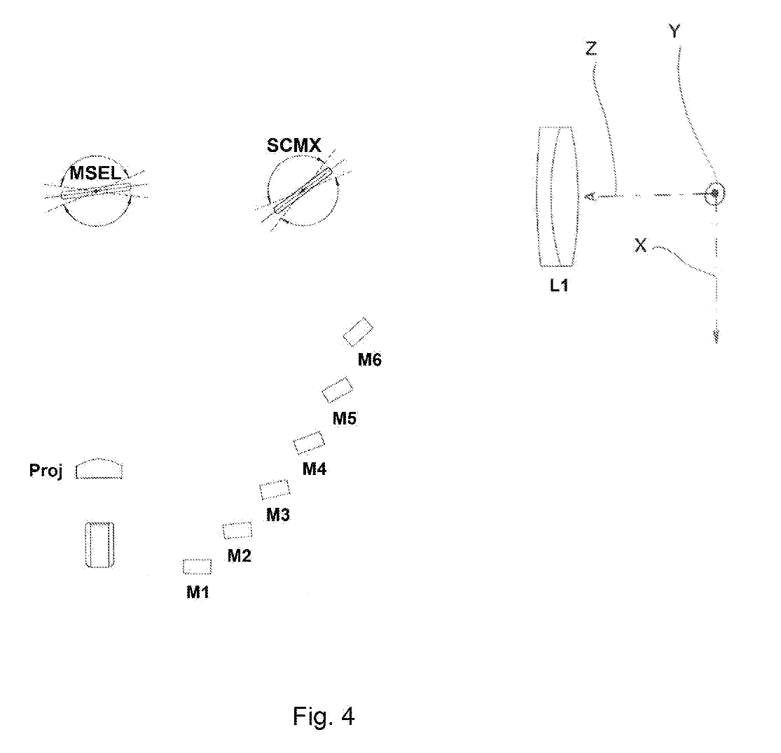

With reference to said figures, and based on what already reported in the introductory part as regards the general architecture of the system, FIG. 4, which is specifically referred to, shows an example of a sample arm of an FD-OCT instrument, such arm being provided with a lens or lens system L1 (of a per se known type) and a tilting mirror MSEL angularly positionable in a certain number of positions, for example six. The lens L1 is centred on the sample, in the case of a human eye the axis of the lens coinciding with the optical axis, indicated as Z. A plane XY may be defined, in the case of the human eye, as the plane tangential to the eye at the incidence point of the optical axis Z. The lens L1 rests parallel to such eye, while the tilting mirror has a rotation axis orthogonal to the plane ZX, and therefore extending along Y (axis coming out of the sheet in the illustration of FIG. 4).

The tilting mirror MSEL is in fact hit by a collimated optical beam F coming from a projector Pr along the direction X. The sample arm further provides for a plurality of mirrors M1 . . . Mk . . . Mn (n=6 in the specific instance) arranged downstream of the tilting mirror MSEL, taking as a reference the path of the optical beam, and oriented so as to intercept the above-mentioned optical beam, each when the beam is reflected in a respective position of the tilting mirror MSEL.

The deviation of the beam in turn reflected by one of the mirrors Mk towards the lens L1, and therefore along the optical axis Z, is provided by a second tilting scanning mirror SCM, controlled so as to tilt in coordination with the first mirror MSEL. In the example the two mirrors are arranged in a substantial alignment along the optical axis Z, while the fixed mirrors M1-M6 are arranged according to an arc shape at progressively smaller distances from the above-mentioned axis, where M1, the first mirror in the sequence, is the closest one to the entering beam segment coming from the projector Pr and is the most distant one from the axis. Going from M1 to M6, besides decreasing the distance from the axis Z, the angle progressively varies, therefore if the first fixed mirror M1 and the optical axis are in a relation of substantial mutual parallelism, the following mirrors M2 . . . M6 are progressively tilted to form a progressively smaller angle between the reflecting face, facing towards the tilting mirrors, and the same optical axis.

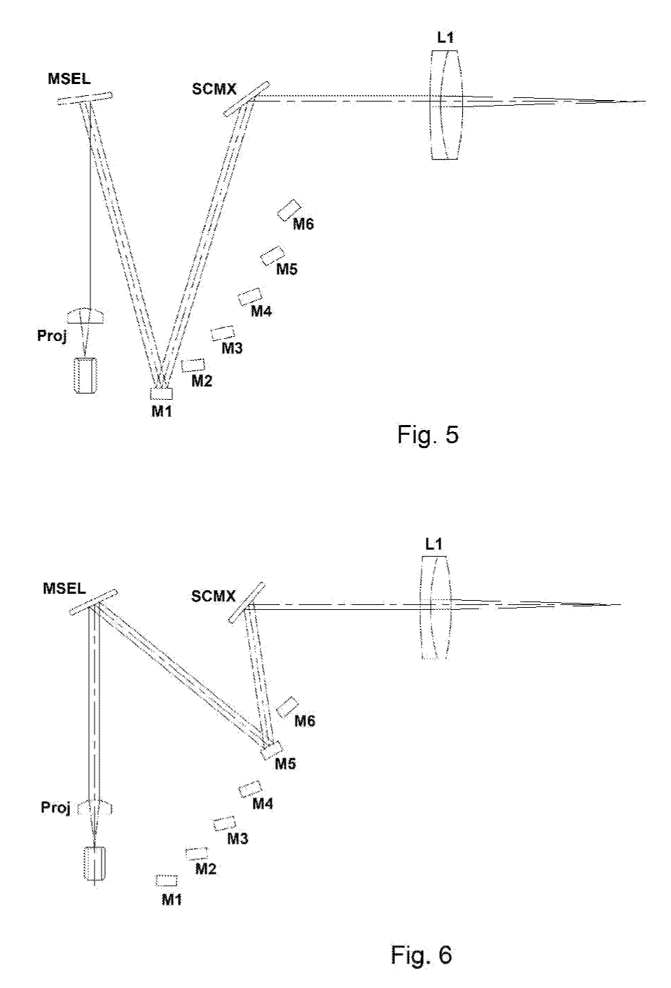

Clearly, depending on the angular position selected for the first tilting mirror MSEL, and correspondingly for the second tilting mirror SCM, optical paths having different lengths are determined for the beam in the sample arm. This will result clearer by examining FIGS. 5 and 6, wherein two examples of optical paths respectively corresponding to position 1 (longer path, the fixed mirror M1 is hit) and position 5 (the fixed mirror M5 is hit) are in fact illustrated.

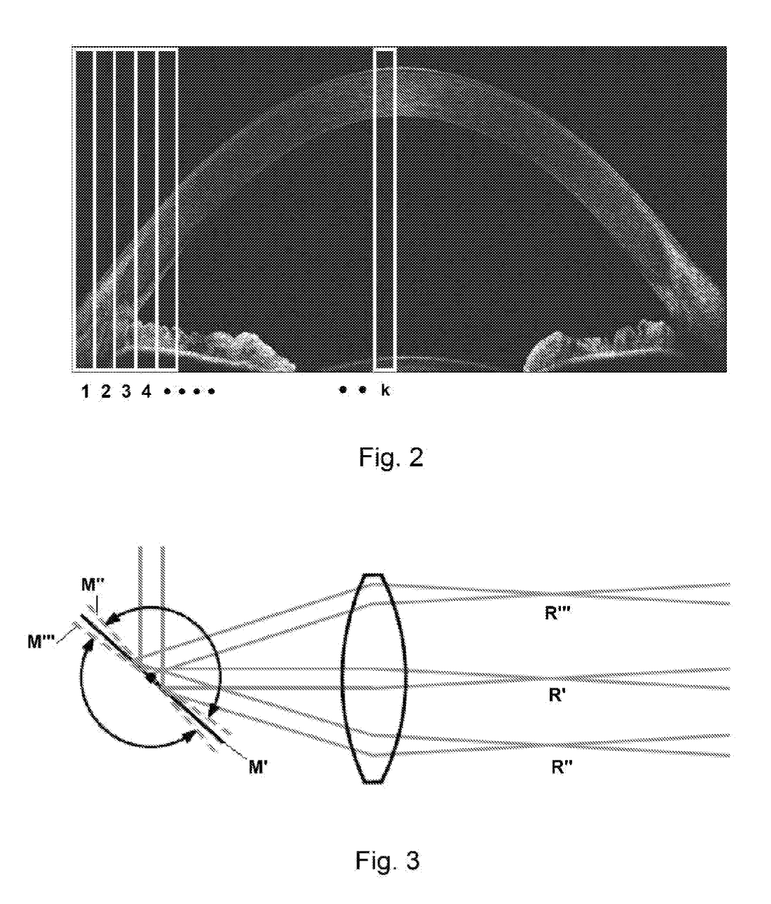

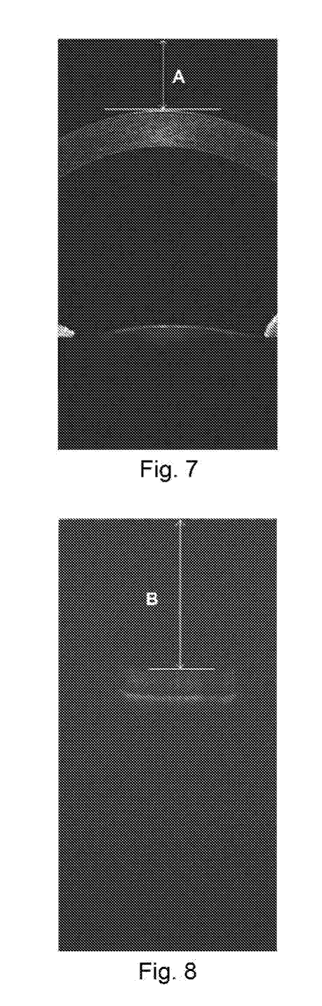

With reference to FIG. 5, the selector mirror MSEL, tilted to an appropriate angular position (position 1) selects a path of maximum length containing the mirror M1 adapted to capture a sample section close to the instrument. In the case of the eye, the mirror M1 will be used to capture the anterior eye segment, obtaining an image as in FIG. 7, which is also connected to that of the previously mentioned FIG. 2. The mirror M6, the one that together with position 6 of the mirror MSEL determines the shortest optical path (not shown), will also be selected when a sample section farther from the instrument is to be captured, that is, more in depth. In the case of the eye, the mirror M6 will be used to capture an image of the retina in particularly “long” eyes, that is, having a high axial extension.

The mirrors M2, M3, M4, M5 (in this latter case reference is to be made to FIG. 6) are selected to capture sample sections which are at progressively greater intermediate depths. For example, the mirror M2 may be used, in the case of an eye, for capturing the crystalline lens and the mirrors M3, M4, M5 for capturing the retina in increasingly “longer” eyes. An image of the retina captured by selecting mirror M5 is shown in FIG. 8.

In the depicted embodiment six paths having different length may be obtained, but such number shall clearly be considered as merely exemplary. In practice, the number of implemented paths, by means of a corresponding number of fixed mirrors and positions of the tilting mirrors, will depend on a compromise between the distances to be measured, the costs, the constructional simplicity, the resolution of the spectrometer or the maximum depth that the OCT system can scan.

Optionally the mirror SCM may be replaced by a pair of mirrors SCMx and SCMy (not shown), tiltable about respective axes orthogonal with each other, so as to obtain a concurrent deviation of the beam in two directions. In any case, the beam finally hits the lens L1 and is focused by the latter at a predetermined distance where the sample to be captured is found. If there are two moving scanning mirrors on axes orthogonal with each other, the appropriate combination of the angular positions occupied in quick succession by the two mirrors will allow carrying out various scanning patterns, known to the man skilled in the art, for example the star-shaped scan of multiple meridians or the raster scan of multiple parallel sections of the object. If only one scanning mirror is provided, it is also possible to envisage a further degree of freedom, that is a further tilting about the axis Z so as to select the angle of the section to be scanned.

Returning to the primary task of the invention, that is to obtain measurements in depth of the distances between the eye interfaces, by taking advantage of the embodiment configuration described above, it is possible to suggest various strategies for measuring the distances between the surfaces of a sample.

A first, simple strategy provides for capturing an image of the sample by selecting each time a different position of the selection tilting mirror MSEL, and then a different mirror Mk, and then another path of different length on the sample arm. If Ml, then M2, M3, M4, M5 and M6 are selected, an image of a sample section close to the instrument will be captured first via Ml, then another one farther away by selecting M2 and so on until capturing the deepest section of the sample via M6. Each time that a mirror Mk is selected the scanning mirror SCM is tilted correspondingly so as to scan a sample section at the selected depth. In order to achieve a fast final measurement, devices for selecting the optical path, scanning and capturing the sample having a correspondingly fast response must be used that the man skilled in the art may easily find. The mirror MSEL may be for example a galvanometric mirror, as well as the scanning mirror SCM; the sensor for collecting the power backscattered by the sample towards the spectrometer may be a high speed line scan camera.

If the sample is an eye, a particularly important measurement in cataract surgery is the distance between the anterior corneal surface and the retina. In this type of surgery this distance is critical for calculating the power of the artificial crystalline lens to be implanted in place of the opacified natural one. By knowing this distance, an optical and geometrical model of the anterior segment and the rated optical and geometrical data of the artificial lenses, it is possible to assess the power of the lens to be implanted into the eye under examination by means of various formulas and methods well known in the literature.

According to the present invention it is possible to measure all the distances between the various intraocular interfaces (anterior and posterior corneal surfaces, crystalline lens surfaces, retina). By way of example, it is now supposed that the axial eye length is to be measured. It is possible to assume that the image of the anterior segment is obtained by using path 1 which includes mirror M1, and that the image of the retina is, on the other hand, obtained using path 5 which includes mirror M5 (reference is therefore made again to what is schematised in FIGS. 5 and 6). From the image of the anterior segment (illustrated as mentioned in FIG. 7) it is then possible to determine the distance A of the anterior corneal surface from the upper edge of the same image, while from the image of the retina (FIG. 8) B is on the other hand determined as the distance of the retinal surface from the upper edge of the image. Then by knowing the difference in the optical path C between the two paths of the sample arm selected respectively for the anterior segment and for the retina, the optical axial length OAL may be determined as:

OAL=C+B−A

Of course, this calculation may be carried out automatically, so that the operator directly obtains the OAL value.

As regards the scans that are performed each time that a different path is selected on the sample arm, a scan may consist for example in 256 A-scans performed on adjacent tissue strips moving the scanning mirror (or the two scanning mirrors, if provided, about their respective axes), or the scanning mirrors may be kept still by repeating many acquisitions of the same tissue strip, or yet a scan on multiple lines on a square area may be performed. In this latter case several A-scans may be captured on an adequately sized square Cartesian grid, for example 16 rows with 16 A-scans each, if the same timing of the line scan is to be maintained.

A reasonable time for scanning both a portion of the anterior segment and a portion of an inner eye structure during the procedure described above is in the order of 10 ms. This time is long enough to collect an amount of radiation on the sensor that is appropriate for obtaining a few hundreds of A-scans, but at the same time it is short enough to prevent artifacts due to eye movement in the range related to an entire B-scan.

In order to determine which is the right path to obtain an image of the retina, a longer time is needed, so that it makes more likely that an eye movement occurs during the attempts of selecting the various paths. The strategy described previously, even though it may appear satisfactory considering also its marked simplicity, is subject to improvements capable of obviating the eye movements of the patient, in particular along axis Z, movements that can in fact occur in the passage from one path to the other and for which the previous formula does not account. In this way it is possible to reduce the incidence of errors which, for example in the measurement of the axial length for determining the power of the lens to be implanted in cataract surgery, may be critical.

A more complex strategy capable of accounting for eye movements may be structured as follows. Path 1 is selected which includes mirror M1 and the anterior segment is captured. Path 2 is then selected which hits mirror M2 and the acquisition goes much deeper. If in the captured image the retina is not detected, path 3 is selected with mirror M3 to capture the image at an even greater depth. Again, if the retina does not appear in the captured image, path 4 is selected with mirror M4. This continues until the k-th path selected allows identifying the retina. Then path 1 is selected again to re-capture an image of the anterior segment and again back to the k-th path to re-capture the retina and so on, alternating acquisitions obtained by selecting with mirror MSEL path 1 and the k-th path. The measurement of interest may then be obtained by N pairs of images of the anterior segment and of the retina captured in an alternating manner thanks to the mirror MSEL, which is rapidly switched between the position suitable for shooting the anterior segment and the position suitable for shooting the retina. The detail of the calculation is described hereinafter.

If upon the i-th acquisition of the pair of images of the anterior segment and the retina Aᵢ is used to indicate the distance of the anterior corneal surface from the upper edge of the image of the anterior segment (FIG. 7), Bᵢ to indicate the distance of the retinal surface from the upper edge of the image of the retina (FIG. 8) and Cᵢ to indicate the difference in the optical path of the two paths of the sample arm selected for the anterior segment and the retina, we find that the optical axial length OALi which may be calculated via the i-th acquisition is:

UALᵢ=Cᵢ+Bᵢ−Aᵢ

If If N acquisitions are considered, an average optical axial length will be obtained from the relation:

$$\overline{OAL} = \frac{1}{N} {\sum }_{i = 0}^{N - 1} {OAL}_{i}$$

Even in this case, the calculation will typically be automated by means of control software implemented with per se simple techniques.

As is known in the literature, from the optical lengths it is possible to obtain the geometrical lengths using the refractive indices of the eye means passed through. The measurement of the distances between the various intraocular structures with equipment as that described above may be carried out in cascade upon acquisition of multiple sections of the anterior segment which allow its three-dimensional measurement or in an ad hoc separate examination uniquely for calculating distances between two or more eye interfaces.

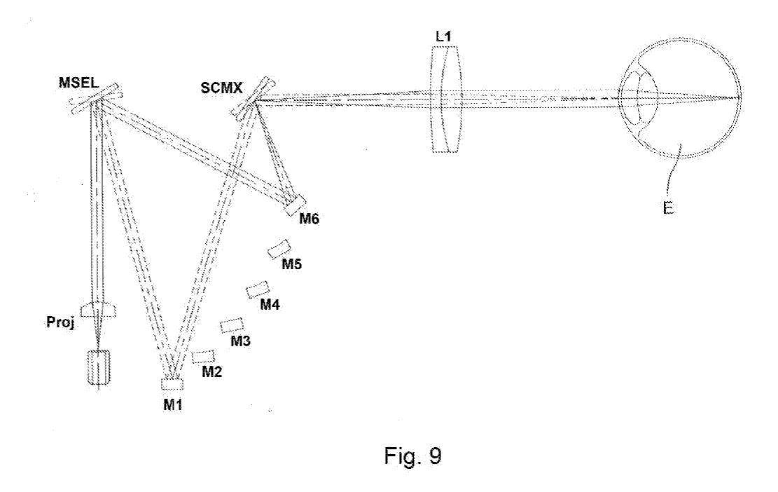

In order to improve the transverse resolution of the images captured at the different eye depths, the mirrors M1, . . . , M6 may be made with curved reflecting surfaces, paying attention to designing the curves so that the focus of the scanning beam coming out of the lens L1 matches the distance at which the scan is to be performed. Such embodiment solution is illustrated in FIG. 9, wherein the dashed line shows the radiation beam when mirror M1 is selected and the solid line shows the beam when mirror M4 is selected. In the first case a portion of the sample close to L1 is to be scanned and the scanning beam focuses this portion; in the second case, on the other hand, a farther portion of the sample is to be scanned and the scanning beam focuses such farther portion, such focusing being enhanced by the different curves of the various mirrors. All of the above is illustrated graphically with even greater clarity by the inclusion, in the illustration, of an eye E being examined.

In this type of interferometry a broadband radiation is used which passes through dispersive components (glass, optical fibres, etc.). The eye also denotes a dispersive behaviour. If the radiation going through the sample arm and that going through the reference arm are not balanced in terms of dispersion, that is they do not pass through the same lengths in glass and/or tissue, there is a deterioration of the instrument's resolution. In view of these considerations, a further advantageous embodiment of the invention provides for compensating the dispersion effect by inserting in the various paths of the same arm elements in glass or an appropriate material having different length. These are capable of making the lengths of the dispersive tracts present on the reference arm and the sample arm identical or very similar to each other, being sized especially considering the lengths of the tracts covered by the radiation in the components of the instrument and also in the eye tissues in a manner independent of the depth at which the path of the sample arm is intended for operation.

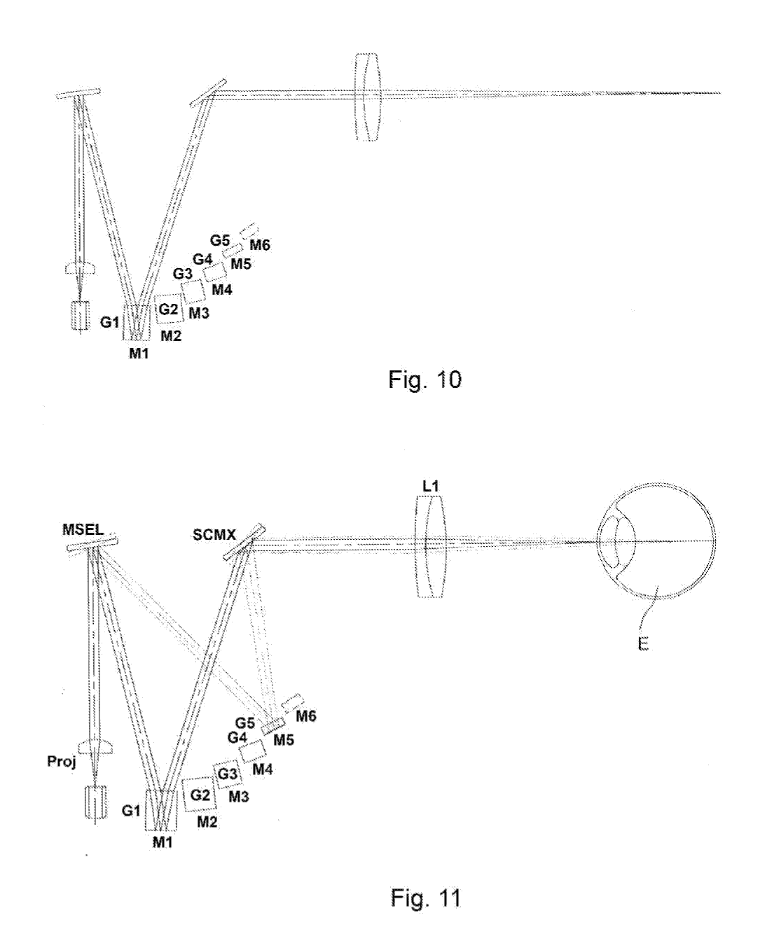

Such embodiment solution is schematised in FIG. 10 where, close to the mirrors M1, M5, there have been placed elements in glass of different lengths G1, . . . , G5. Mirror M6, on the other hand, does not have a corresponding element in glass. With this type of configuration also the reference arm will have to be provided with a sufficiently long element in glass which has the same dispersion of the eye means going from the cornea to the deep area of which the image is captured when the path with mirror M6 is activated.

FIG. 11 finally shows an embodiment solution wherein the compensation of the dispersion is combined with the adoption of mirrors having appropriate curves in order for the focus of the scanning beam coming out of the lens L1 to match the distance at which the scan is to be performed. In practice, the embodiments of FIG. 9 and FIG. 10 are here associated to each other.

The present invention therefore provides a fully satisfactory response to the predetermined task, combining a precise and reliable functional result with a simple and an actually feasible and structurally simple configuration at low costs, also from a management and maintenance standpoint.

With an N number of different paths on the sample arm, selectable thanks to a tilting mirror which with a small and quick tilting is driven from one to the other of N angular positions useful for acquisition at the desired depth, the acquisition may go from one depth to the other, and with alternating acquisitions between two desired depths, obtained by selecting alternatively the two suitable paths of the sample arm, the measurements of the distance between the eye structures of interest present in images relating to different depths may be repeated many times in a short time interval. In this way, the measurement of the distance between the eye structures is robust, that is, safe and reliable, in spite of any movements of the eye being examined.

Such a result is obtained without using multiple reference arms/paths, either dichroic separation of the beam coming from the sample, or the need of bulky and costly sensors with a high number of pixels, or yet longitudinal movements which are difficult to fine tune (the movement in bursts of the tilting mirror MSEL in predetermined positions ensures the desired precision over time without particular problems and at significantly lower management costs).

The preceding solutions only represent illustrative examples and should not be considered as the only ones adapted to the task. Various combinations of the conceptual solutions illustrated hereinabove shall be considered as implicitly understood by the man skilled in the art. The present invention, however, has been described thus far with reference to its possible exemplary embodiments. It must be understood that there may exist other embodiments, within the scope of overall optical configurations different from that disclosed herein and integrated by additional components/functionalities, belong to the same inventive scope, all falling within the scope of protection of the attached claims.

1. A optical coherence tomography system comprising: —a broadband light radiation source (LBS); —a reference optical arm (RA); —a sample optical arm (SA) comprising movable scanning means (SCM) for scanning a sample, adapted to receive the light radiation emitted by said source to illuminate with a scanning beam a portion of the sample corresponding to a position of the scanning means (M), generating a radiation hitting along an optical axis (Z) a surface of the same sample, and to collect the backscattered radiation from the sample; a signal detection arm (MA) with at least one sensor adapted to reconstruct the spectrum of the signal resulting from the recombination of the radiation collected by said reference arm (RA) and by said scanning means (SCM) of the sample arm (SA); beam splitter means adapted to permit the passage of the radiation from the source (LBS) to the sample arm (SA) and to the reference arm (RA), and from these to the detection arm (MA); and a control and processing unit (CUP) adapted to control the above mechanical and electronic components, to transform said spectrum in a reflectivity profile of the illuminated sample portion, and to generate an image of the sample by juxtaposing a number of profiles each corresponding to a sample portion and obtained further to a displacement of said scanning means; wherein said sample optical arm (SA) comprises selection means (MSEL) tiltable between among two predetermined positions to selectively deviate said scanning beam over at least two respective and alternative optical paths having different lengths, adapted to collect information relative to portions of the sample at different depths along said optical axis (Z).

2. The system according to claim 1, wherein said selection means comprise a tilting selection mirror (MSEL) tiltable between said at least two predetermined positions and at least two corresponding fixed mirrors (M1 . . . Mk . . . Mn) arranged downstream of the tilting mirror (MSEL), so as to receive said scanning beam and deviate it towards the scanning means (SCM), each fixed mirror when the beam is reflected by either position of the tilting mirror (MSEL) to selectively define respective optical paths.

3. The system according to claim 2, wherein said selection mirror (MSEL), said scanning means (SCM) and said sample are substantially aligned along said optical axis (Z), said fixed mirrors (MK) being arranged according to an arc shaped distribution at distances progressively reduced with respect to said axis starting from a first fixed mirror (M1) closer to an entering beam segment coming from said source (LBS).

4. The system according to claim 3, wherein the angle between a fixed mirror reflecting faces facing towards said selection mirror (MSEL) and the optical axis becomes progressively reduced starting from the fixed beam (M1) closer to the entering beam segment.

5. The system according to claim 2, wherein said scanning means comprise a scanning mirror (SCMy) tilting around an axis coplanar and parallel with a tilting axis of said selection mirror (MSEL).

6. The system according to claim 5, comprising a pair of scanning mirrors tilting around respective axis orthogonal with each other, so as to obtain a deviation of the scanning beam, for each optical path, in two distinct directions.

7. The system according to claim 2, wherein said fixed mirrors (M1 . . . Mk . . . MN) have curved reflecting faces adapted to focus the scanning beam in accordance with the sample depth to the scanning of which each mirror is intended.

8. The system according to claim 2, further comprising for each of said fixed mirrors (M1 . . . MK . . . Mn) compensating elements adapted to make mutually uniform the lengths of the dispersive segments in said reference arm (RA) and in the respective paths in the sample arm (SA).

9. The system according to claim 8, wherein said compensating elements comprise glass elements (G1 . . . G5) of different size arranged close to respective fixed mirrors (M1 . . . M5).

10. A optical coherence tomography method wherein: —in a sample optical arm a sample is scanned by collecting a backscattered radiation following a broadband lighting radiation hitting with a scanning beam along an optical axis portions of a surface of the same sample; —a sensor reconstructs the spectrum of the signal resulting from the recombination of the radiation collected by an optical reference arm and by the scanning; said spectrum is transformed into a reflectivity profile of the illuminated sample portion, and an image of the sample is generated by juxtaposition of a number of profiles each corresponding to a sample portion and obtained as the scanning advances portion after portion; wherein said optical arm said scanning beam is selectively deviated over at least two respective and alternative optical paths having different lengths to collect information relative to portions of the sample at different depths along said optical axis.

11. The method according to claim 10, wherein said alternative optical paths are obtained by tilting a selection tilting mirror (MSEL) between at least two predetermined positions.
